(19) United States
(12) Patent Application Publication
(10) Pub. No.: US 2026/0076686 A1
(43) Pub. Date: Mar. 19, 2026

(54) Resection Guide to Alter Distal Femoral Flexion

(71) Applicant: Howmedica Osteonics Corp., Mahwah, NJ (US)

(72) Inventors: Kerianne Coulon, Hoboken, NJ (US); Justin Moidel, Verona, NJ (US)

(21) Appl. No.: 19/329,848

(22) Filed: Sep. 16, 2025

Related U.S. Application Data
(60) Provisional application No. 63/695,420, filed on Sep. 17, 2024.

(51) Int. Cl. A61B 17/17

(52) U.S. Cl. CPC A61B 17/1764

(57) ABSTRACT

A cutting guide is provided for resecting a bone to receive an implant and includes a body configured to be positioned adjacent to the bone and a slot extending through the body configured to receive a cutting edge for resecting the bone. When a rod is disposed in an intramedullary canal of the bone and an opening of the body is passed over the rod, the body is adjustable such that a plane through the slot changes relative to the bone as the body is adjusted. Additionally, the plane through the slot is transverse to a central axis extending through a length of the bone, and the cutting guide is configured such that when the body is received on the rod anchored within the bone, the plane represents a path of resection through the bone, and adjustment of the body alters the path of resection through the bone.

### CROSS-REFERENCE TO RELATED APPLICATIONS

This application claims the benefit of the filing date of United States Provisional Patent Application No. 63/695,420 filed September 17, 2024, the disclosure of which is hereby incorporated herein by reference.

### BACKGROUND OF THE INVENTION

Revision total knee arthroplasty (TKA) procedures involve the replacement of portions of one or more of the patella, femur and tibia with artificial components. In particular, at least part of a procedure may involve resection of a distal portion of the femur to be replaced with artificial components. There are several types of knee prostheses known in the art. One type is sometimes referred to as a “resurfacing type.” In procedures that utilize these prostheses, the articular surface of the distal femur is “resurfaced” with condylar-type articular bearing components. These knee prostheses are intended to provide adequate rotational and translational freedom in the knee joint and require minimal bone resection to accommodate such prostheses within the boundaries of the available joint space. Additionally, the femoral components are usually provided with an intramedullary (IM) stem option.

However, those skilled in the art will appreciate that revision TKA procedures are difficult because the type and location of cavernous defects, when present, make it difficult to match the exterior surfaces of the tibia and femur to the interior surfaces of the prosthesis, the femur and tibia must be resected with reference to the IM canal, and the use of multiple templates and guides during the course of the procedure makes it very difficult to keep all the cuts in proper alignment relative to the IM canal.

Additionally, during revision TKA procedures, it is desirable to balance the knee in both flexion and extension. For example, the femur is typically positioned in flexion relative to a mechanical axis in order to reduce the flexion gap and support a more well-balanced knee. However, there is an absence of a consistent and predictable approach for controlling the amount of flexion added prior to resecting the distal femur. Further, existing techniques intended to account for the flexion gap fail to do so in a controlled and consistent manner.

Accordingly, a need exists for improved instrumentation and methods for preparation of a long bone for receipt of an implant.

### BRIEF SUMMARY OF THE INVENTION

In a first example of a first aspect, the present disclosure relates to a cutting guide for resecting a bone to receive an implant. The cutting guide comprises a body configured to be positioned adjacent to the bone, a slot extending through the body and configured to receive a cutting edge, the cutting edge being configured to resect the bone, and an opening extending though the body, the opening being configured to receive a rod. The cutting guide is further defined wherein when a rod is disposed in an intramedullary canal of the bone and the opening of the body is passed over the rod, the body is adjustable such that a plane through the slot changes relative to the bone as the body is adjusted. The cutting guide is further defined wherein the plane through the slot is transverse to a central axis extending through a length of the bone, and wherein the cutting guide is configured such that when the body is received on the rod anchored within the bone, the plane represents a path of resection through the bone, and adjustment of the body alters the path of resection through the bone.

In a second example, the first example of the first aspect is further defined wherein the body includes a bone facing surface and an articulation surface opposite the bone facing surface. In a third example, the first example of the first aspect is further defined wherein the bone is a femur and the body is configured to be positioned on the femur such that the cutting edge cuts in an anterior-posterior direction. In a fourth example, the first example of the first aspect further comprises a hole extending through the body, the hole being configured to receive a pin anchorable to the bone. In a fifth example, the fourth example of the first aspect is further defined wherein the hole is configured to be positioned adjacent a proximal end of the body and remote from the opening. In a sixth example, the first example of the first aspect further comprises a reference slot extending through the body and configured to receive the cutting edge for resecting the bone, wherein the reference slot is configured to be positioned adjacent an anatomical point on the bone such that the anatomical point is within a reference plane parallel to a plane through the slot. In a seventh example, the sixth example of the first aspect is further defined wherein the bone is a femur and the anatomical point is a medial epicondyle of the femur. In an eight example, the first example of the first aspect is further defined wherein the body comprises a flexible material, the body configured to flex as the body is adjusted relative to the bone. In a ninth example, a system includes the cutting guide of the first example of the first aspect and the system is further defined wherein the rod is configured to flex sufficiently to alter an alignment of the rod when disposed in an intramedullary canal of a long bone. In a tenth example, the first example of the first aspect is further defined wherein the rod is a reamer.

In a first example of a second aspect, the present disclosure relates to a method for preparing a bone for receiving an implant. The method comprises reaming a hole through the bone; anchoring a rod within the hole; positioning an opening of a cutting guide over the rod and onto an end portion of the bone such that cutting guide is in a first orientation relative to the bone; and moving the cutting guide and a portion of the rod positioned through the opening relative to the bone into a second orientation relative to the bone, wherein in the first orientation, a slot of the cutting guide is coincident with a first plane and in the second orientation, the slot of the cutting guide is coincident with a second plane, each of the first plane and the second plane being defined relative to the bone, and wherein the first plane defines a cutting path through the bone at a different slope in an anterior-posterior direction than the second plane.

In a second example, the first example of the second aspect is further defined wherein the bone is a femur and the hole through the bone is an intramedullary canal. In a third example, the first example of the second aspect further comprises, after the moving step, comparing the cutting path through the bone for the cutting guide in the first orientation and the second orientation and determining a desired cutting path through the bone. In a fourth example, the third example of the second aspect is further defined wherein the articular surface of the cutting guide includes a reference slot, a third plane passing through the reference slot being parallel to the first and second planes passing through the slot in the first and second orientations, respectively. In a fifth example, the first example of the second aspect further comprises, after the moving step, anchoring the cutting guide to the bone with a pin extending through a pin hole of the cutting guide. In a sixth example, the first example of the second aspect further comprises, after the moving step, cutting a portion of the bone through the slot of the cutting guide when the cutting guide is in the first or second orientation. In a seventh example, the first example of the second aspect is further defined wherein the bone is a first bone, and the method further comprises, after the moving step, assessing a flexion space between the first bone and a second bone in the first orientation and in the second orientation, wherein the first bone is a femur and the second bone is a tibia. In an eight example, the seventh example of the second aspect is further defined wherein assessing the flexion space includes determining a cutting guide orientation having a minimal flexion space between the femur and the tibia. In a ninth example, the first example of the second aspect is further defined wherein moving the cutting guide further comprises internally and/or externally rotating the cutting guide about a longitudinal axis of the rod.

### BRIEF DESCRIPTION OF THE DRAWINGS

FIG. 1A is a first perspective view of a bone preparation system according to an embodiment of the present disclosure;

FIG. 1B is a second perspective view of the bone preparation system of FIG. 1A;

FIG. 2A is a front view of a flexible shell of the bone preparation system of FIG. 1A;

FIG. 2B is a rear view of the flexible shell of FIG. 2A;

FIG. 2C is a side view of the flexible shell of FIG. 2A;

FIG. 2D is a first enlarged view of the flexible shell of FIG. 2A;

FIG. 2E is a second enlarged view of the flexible shell of FIG. 2A;

FIGS. 3A-3C show steps in a method of using a bone preparation system according to an embodiment of the present disclosure; and

FIGS. 4A-4C show steps in another method of using a bone preparation system according to an embodiment of the present disclosure.

### DETAILED DESCRIPTION

As used herein, the terms “about,” “generally,” and “substantially” are intended to mean that slight deviations from absolute are included within the scope of the term so modified. To aid the Patent Office and any readers of any patent issued on this application in interpreting the claims appended hereto, Applicant notes that it does not intend any of the appended claims or claim elements to invoke 35 U.S.C. § 112(f) unless the words “means for” or “step for” are explicitly used in the particular claim.

As used herein, when used in connection with the human body, the term “proximal” means closer to the heart, and the term “distal” means further from the heart. As used herein, the terms “substantially,” “generally,” “approximately,” and “about” are intended to mean that slight deviations from absolute are included within the scope of the term so modified.

In one aspect, the present disclosure relates to an improved system for preparing a bone to receive an implant, and specifically for preparing a femur to receive a femoral implant during an initial or revision total knee arthroplasty (TKA) procedure. Although the embodiments described herein and depicted in the Figures are applicable to a distal femur, it should be appreciated that the principles of the present disclosure may be applied to a proximal tibia, the fibula or other long bones in the body.

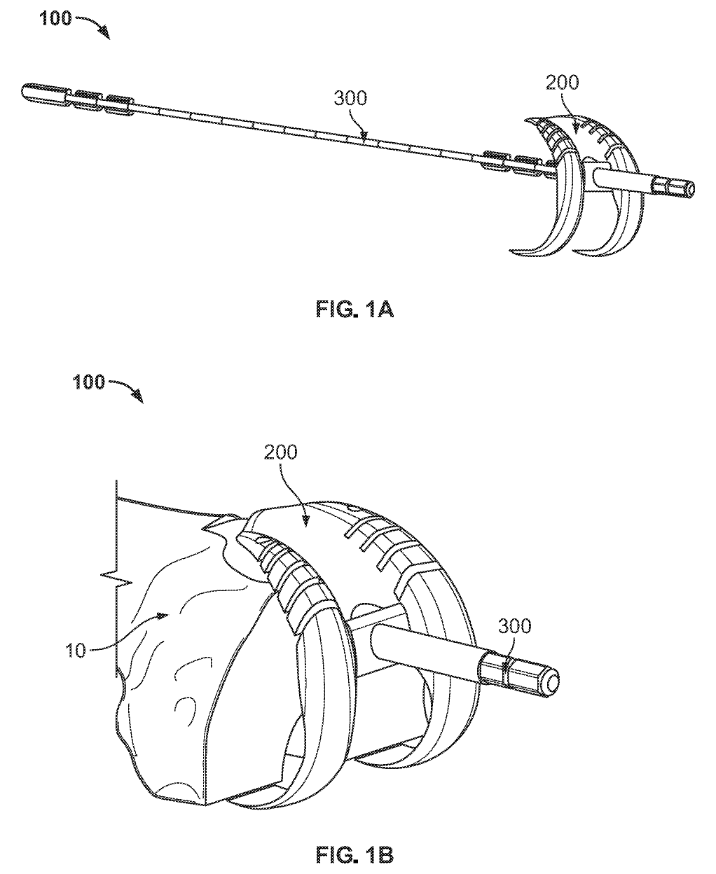

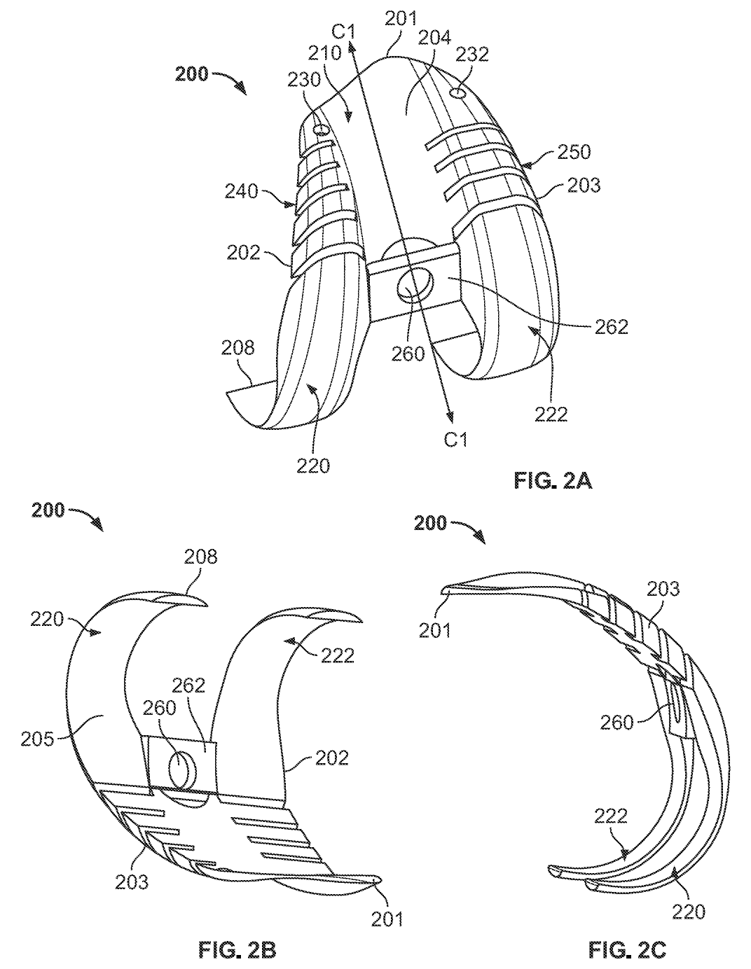

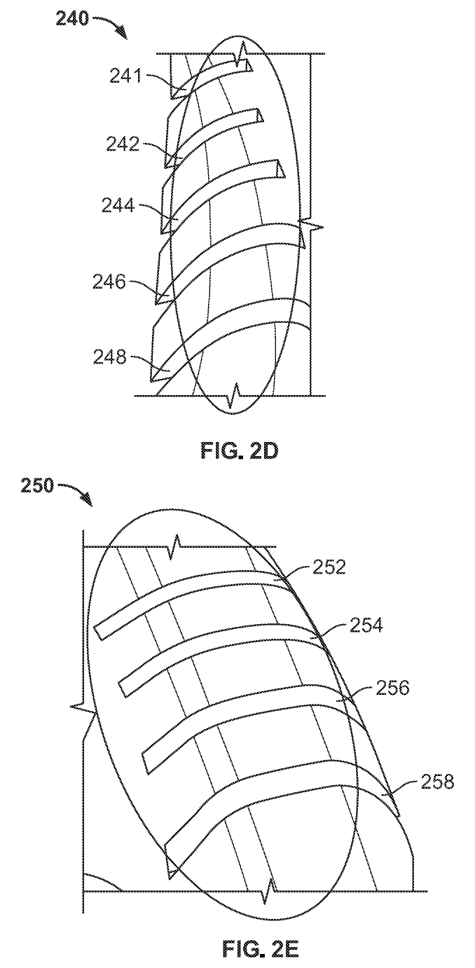

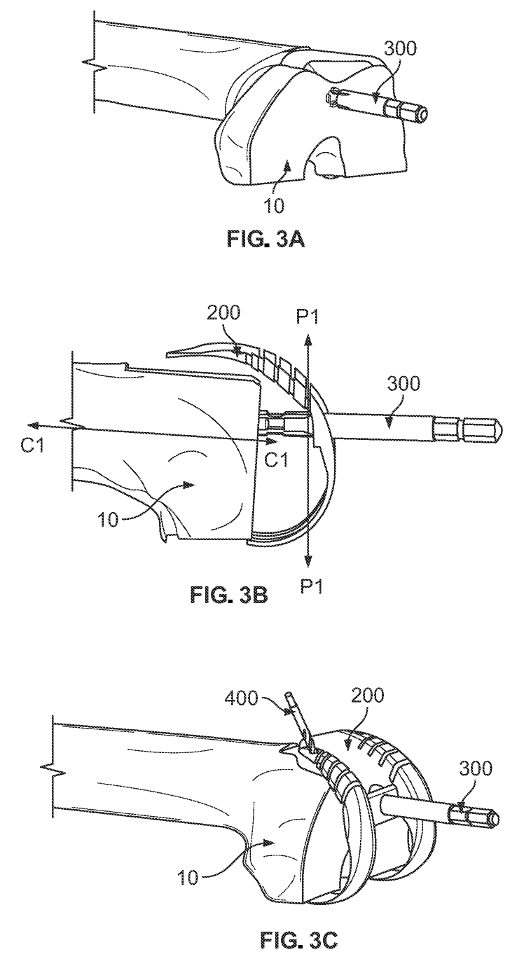

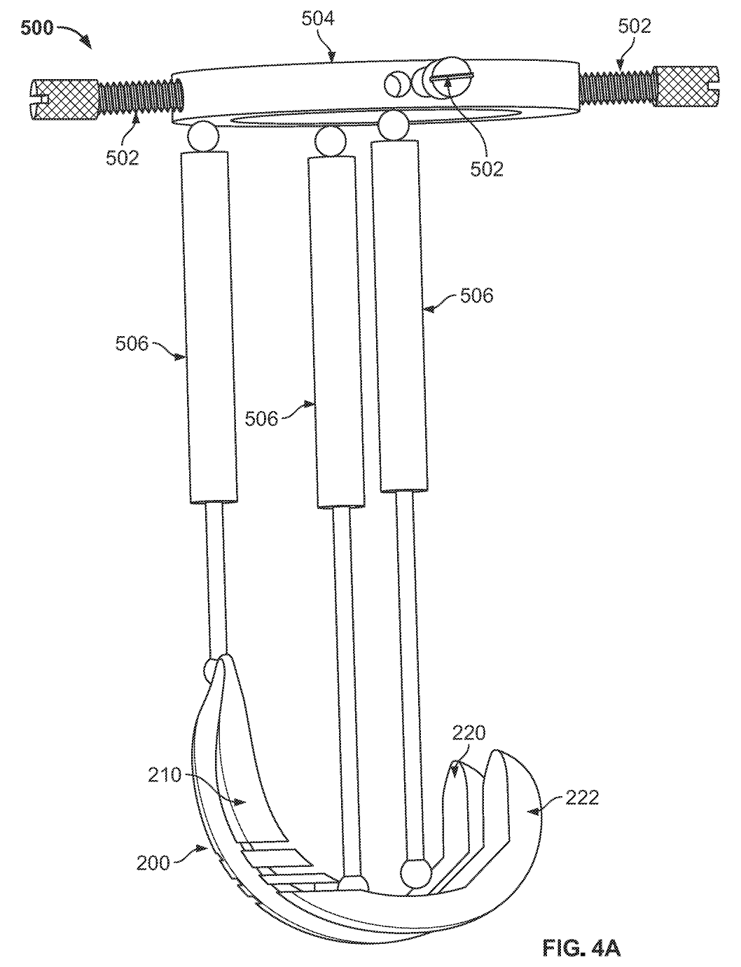

FIGS. 1A-2E illustrate a bone preparation system 100 according to one embodiment of the present disclosure. In one example, system 100 is used for determining and preparing a planned resection of a portion of a distal femur 10 to receive a femoral implant. System 100 is used in a revision TKA procedure to allow a user to control the amount of flexion in the knee based on controlled resection of the distal femur 10, as well as allowing the user to determine and minimize a flexion gap of the knee. Additionally, system 100 may allow the user to determine and assess a distal gap of the knee in extension. Further, system 100 allows a user to determine whether or not a specific angle of resection prevents the user from implanting a stem component within the intramedullary (IM) canal of the distal femur 10. System 100 includes a flexible shell 200 and a flexible IM rod 300. In some embodiments, system 100 also includes one or more bone fixation pins 400, as shown in FIG. 3C, for example. System 100 allows for a user to determine and assess a distal gap of the knee in extension when shell 200 is pinned to the femur and rod 300 is absent from the joint. Shell 200, rod 300, and pins 400 may be comprised of titanium alloy, cobalt chrome, or any other commonly used metal alloys, such as stainless steel. We now turn to the various components of the system. It should be appreciated that individual components of the system are also contemplated as individual standalone devices.

Shell 200 of system 100 is shown in isolation in FIGS. 2A-2E. Shell 200 includes a base portion 210 and posterior legs 220, 222 extending from base portion 210. Shell 200 further includes pin holes 230, 232, a plurality of slots 240, 250, and a flexible IM rod hole 260 each extending through base portion 210. As discussed further below, shell 200 is designed to be anchored to a distal end portion of a femur during a revision TKA procedure. Shell 200 is designed to have increased flexibility due a combination of its material and reduced thickness throughout base portion 210 and posterior legs 220, 222. For example, shell 200 may be comprised of 17-4 PH stainless steel.

Shell 200 includes first pin hole 230 and second pin hole 232 extending through front surface 204 to rear surface 205 of shell 200. Pin holes 230, 232 are disposed near first end 201 of shell 200 and, in the depicted embodiment, are each configured to receive a cylindrical bone fixation pin 400 that is used to anchor shell 200 to a portion of the distal femur 10, as discussed further below. In variations, pin holes 230, 232 may comprise different shapes corresponding to alternative designs or shapes of bone fixations pins 400. First pin hole 230 is disposed through a first side of shell 200, the first side of shell 200 being defined from the first side surface 202 to approximately the central longitudinal axis C1 of shell 200. Second pin hole 232 is disposed through a second side of shell 200, the second side of shell being defined from the second side surface 203 to approximately the central longitudinal axis C1 of shell 200. In some examples, and as depicted, first pin hole 230 has dimensions that match those of second pin hole 232, such dimensions including the circumference, angle, width, and height through the shell 200. In variations, shell 200 may include more or fewer pin holes than shown in the depicted embodiment.

Shell 200 further includes a plurality of slots 240, 250 each extending through a front surface 204 to rear or patient facing surface 205 of the base portion 210 of shell 200. Further, plurality of slots 240, 250 includes a first group of slots 240 and a second group of slots 250 opposite the first group of slots 240. First group of slots 240 extend from a first side surface 202 of shell 200 laterally towards, but are spaced apart from, a central longitudinal axis C1 extending through shell 200, and second group of slots 250 extend from a second side surface 203 of shell 200 also laterally towards, but are spaced apart from, the central longitudinal axis C1. In the depicted embodiment, first group of slots 240 are designed to match second group of slots 250 such that a respective slot from each grouping matches a respective slot from the other grouping. For example, respective slots 242, 252 from the first and second groups of slots 240, 250 may be designed to have the same dimensions, such as the width, height, and length through shell 200. Therefore, the discussion below for first group of slots 240 should also be understood as representative of second group of slots 250 as well. However, first group of slots 240 additionally includes a reference slot 241, referred to as the Medial Epicondyle (ME) slot when used in system 100. Specifically, reference slot 241 may be referred to as the ME slot when shell 200 is attached onto a portion of the distal femur 10, as discussed further below. In some alternative variations of the system, the shell may also include an ME slot within second group of slots 250. Although not explicitly described, it should be appreciated that variations may have one or more differences between the first and second groups of slots.

An enlarged view of first group of slots 240 is shown in FIG. 2D. First group of slots 240 includes an ME slot 241, a first slot 242, a second slot 244, a third slot 246, and a fourth slot 248. However, while ME, first, second, third, and fourth slots 241, 242, 243, 244, 245 are shown and described below, shell 200 may include only one or more of these slots or may include additional slots. ME slot 241 is near first end 201 of shell 200 and fourth slot 248 is near a first posterior leg 220 that extends to a second end 104 of shell 200. ME slot 241 extends shortest laterally through shell 200 to an end proximate the central longitudinal axis C1 extending through shell 200. Fourth slot 248 extends furthest laterally through shell 200. First, second, and third slots 242, 244, 246 each extend laterally through shell 200 a distance in between that of ME slot 241 and fourth slot 248. Each slot of the first group of slots 240 is configured to receive a cutting tool for resecting a portion of the distal femur 10, as discussed further below. In the depicted embodiment, in a direction along the central longitudinal axis C1 extending through the shell 200, ME slot, first, second, third, and fourth slots 241, 242, 244, 246, 248 are evenly spaced apart by 5-millimeter increments. As discussed further below, this design allows for varying depths of resection for a portion of the distal femur 10 when used with system 100. For example, fourth slot 248 corresponds to the distal femur 10 initial resection depth of 5 mm, third slot 246 corresponds to a resection depth of 10 mm, second slot 244 corresponds to a resection depth of 15 mm, and first slot 242 corresponds to a depth of 20 mm. While a distance between each slot of the first group of slots 240 is shown as equal to 5 mm, in variations, this distance may be within a range of 1 mm to 10 mm. In some variations, the length of each slot of the first group of slots 240 may vary from that shown, in absolute terms or relative to another slot. Additionally, the distance between each slot of the first group of slots 240 does not need to be equal and can vary between each respective slot.

Shell 200 further includes a first posterior leg 220 and a second posterior leg 222 each extending from base portion 210 to a second end 208 of shell 200 opposite first end 201. First leg 220 extends from base portion 210 adjacent first side surface 202 of shell 200, and second leg 222 extends from base portion 210 adjacent second side surface 203 of shell 200. Legs 220, 222 each include a generally rounded, monolithic surface extending from base portion 210 in a direction opposite front surface 204 of shell 200. Legs 220, 222 are designed to contact a portion of the distal femur 10 when shell 200 is attached to the distal femur 10, as discussed further below.

Shell 200 further includes a bridge portion 262 spanning legs 220, 222 and spaced apart from base portion 210. Flex IM rod opening 260 extends through bridge portion 262 from front surface 204 to a rear surface 205 of shell 200. Opening 260 may be substantially circular. In variations, a shape of opening 260 may be non-circular as appropriate to accommodate a reaming tool, such as a boss reamer or IM reamer, and/or stem trial component. Opening 260 is designed to receive and align flexible IM rod 300 disposed at least partially within the IM canal of the distal femur 10, as discussed further below. Bridge portion 262 may include engagement mechanisms to prevent rotation of rod 300 while positioned through opening 260. In other variations, bridge portion 262 may simply include an opening 260 without any engagement mechanism.

In another aspect, the present application relates to a kit including a combination of components including at least one component of the system 100. In some embodiments, a kit may include one or more components from system 100. For example, the kit may include a shell 200, IM canal reamer 300 and a plurality of pins 400. In some embodiments, a kit may include two or more of the system components. For example, a kit may include two or more shells 200. In some variations of this kit, two or more shells having different sizes or other characteristics, such as slot configurations, may be included.

Any combination of system components may also be included in a single package or in individual packages which may later be brought together to create a kit. When more than one package is used, any one package in such kit may include one or more of the components of the kit. It is also contemplated that a kit may include any combination of system components along with one or more additional instruments used to place such securement devices in a patient. In other examples, the kits contemplated herein may be accompanied by an instruction manual on how to perform one or more of the methods of using the contents of the kit.

In another aspect, the present disclosure relates to a method for performing a surgical procedure for preparing a bone to receive an implant, and specifically for preparing a distal femur to receive a femoral implant during a revision TKA procedure. As explained elsewhere in the present application, it should be appreciated that the system and components thereof as contemplated by the present disclosure are not limited to use in revision TKA procedures. And, to the extent the methods below are described with respect to a revision TKA procedure, such description is for purposes of illustration. It should be understood that the following operations do not have to be performed in the exact order described below. Instead, various steps may be handled in a different order or simultaneously. Steps may also be omitted or added unless otherwise stated herein.

In one embodiment, a method for preparing a bone for receiving an implant is as illustrated in FIGS. 3A-3C and begins with a first step of reaming an IM canal of a distal femur 10. As previously discussed, an implant is configured for surgical procedures in either the left or right distal femur. In one non-limiting example, the IM canal of a femur is progressively reamed by an IM reamer until cortical chatter is achieved.

In a second step, the IM reamer is removed from the IM canal of the distal femur 10, and a flexible IM rod 300 is positioned within the reamed IM canal, as best shown in FIG. 3A. In other examples, rod 300 may not be positioned within the distal femur 10 and the IM reamer may remain firmly seated in the IM canal, and shell 200 may be positioned over the IM reamer as described in the third step further below. In still further examples, the IM rod 300 may not be positioned within the distal femur 10 and a stem bushing or stem trial may be positioned in the reamed IM canal, and shell 200 may be positioned over the stem bushing also as described in the third step further below. The stem bushing may include a through hole extending though its center and designed to receive an end or side cutting reamer for preparation of the stem in the distal femur 10. The end cutting reamer designed to be positioned through the stem bushing may, in some examples, have a diameter of 11 mm, 14 mm or 17 mm.

In a third step, a flexible shell 200, via opening 260, is positioned over rod 300 and onto the distal femur 10, as best shown in FIG. 3B. Shell 200 is positioned such that rod 300 extends through a flexible IM rod hole 260 and rear surface 205 of shell 200 is adjacent to and faces the distal femur 10. Specifically, a rear surface 205 of a base portion 210 of shell 200 is positioned adjacent an anterior portion of the distal femur 10, and rear surface 205 of the first and second legs 220, 222 are positioned adjacent a posterior portion of the distal femur 10. Further, rod 300 holds shell 200 in position while the knee joint is in flexion, for example, prior to anchoring shell 200 to the distal femur 10 as described in the fourth step further below.

In a fourth step, shell 200 is adjusted, e.g., rotated in the sagittal plane and about a central longitudinal axis C1 extending through the distal femur 10, such that ME slot 241 of shell 200 is positioned to be aligned with the medial epicondyle (ME) of the distal femur 10. Additionally, adjustment of shell 200, when positioned over rod 300, causes rod 300 to flex within the IM canal. Specifically, rod 300 is designed to be able flex, allowing the rod 300 curvature to match the contour of the IM canal, and aiding in the ability to adjust shell 200 relative to the axis C1 extending through the distal femur 10. Shell 200 is flexed in order to align the slots in a desired position relative to the distal femur 10. For example, shell 200 is designed with slightly shortened legs 220, 222 to allow a user to assess flexion space in the knee joint prior to committing to a resection of the distal femur 10. Shell 200 is assessed by the user to determine whether which, if any, of slots 240, 250 create a desired flexion or resection angle, i.e., a desired resection cut line on a distal end surface of the distal femur 10. The desired flexion or resection angle may be described as an angle relative to a plane that is normal to the central longitudinal axis C1 of the femur. For example, as shown in FIG. 3B, when ME slot 241 is aligned with the ME of the distal femur 10, shell 200 is in a desired position such that first slots 242, 252 are positioned along a first plane P1 extending through a portion of the distal femur 10 in the anterior/posterior direction. A user considers whether a satisfactory alignment and desired resection cut line has been established, or if shell 200 requires readjustment. Additionally, at this time, the user evaluates the flexion gap between legs 220, 222 of shell 200 and a proximal portion of the tibia. For example, a tibial baseplate trial or tibial template and insert trial may be positioned on the tibia to assess whether the flexed femoral positioning of shell 200 sufficiently closes the posterior femoral gap, therefore closing the flexion gap. After satisfactory alignment has been determined, first and second pins 400 are each respectively anchored into a portion of the distal femur 10 through first and second pin holes 230, 232 of shell 200, as best shown in FIG. 3C. In this manner, the anchored pins 400 allow for shell 200 to be attached to a portion of the distal femur 10 with a desired alignment of the slots 240, 250. In other embodiments, shell 200 may include additional pin holes or openings on base portion 210 or legs 220, 222 to respectively receive additional anchored pins 400.

In a fifth step, once shell 200 is in the final position, resections through any of the respective slots 240, 250 are made to prepare the distal femur 10 for receiving a femoral implant. After this step, additional resections may be made, with or without shell 200 anchored to the distal femur 10, to further prepare the distal femur 10 for receiving the femoral implant.

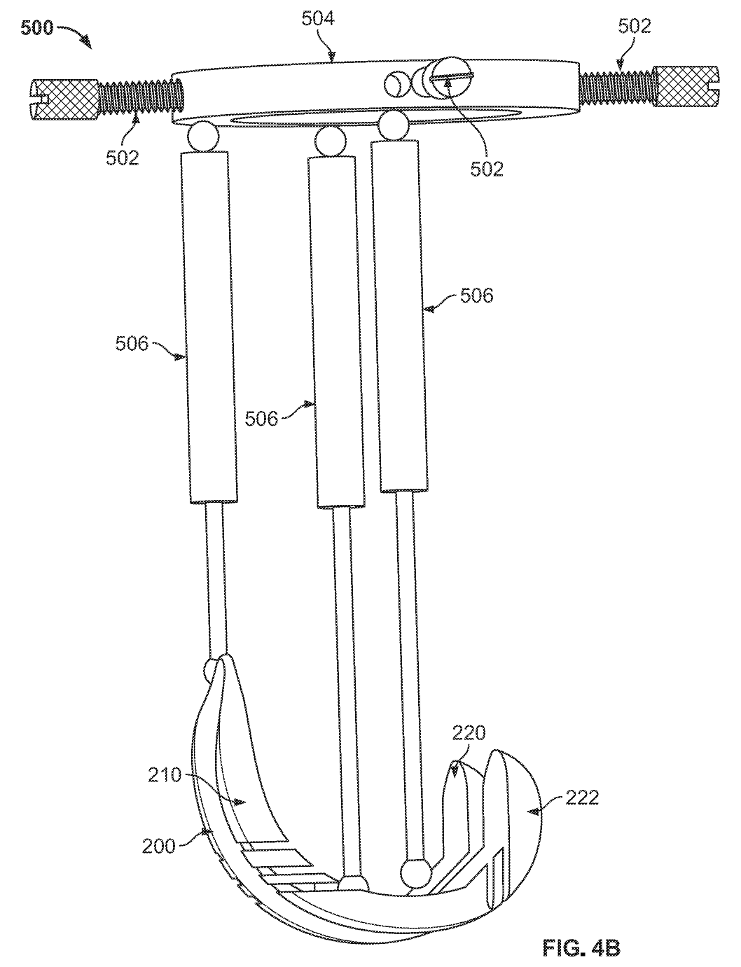

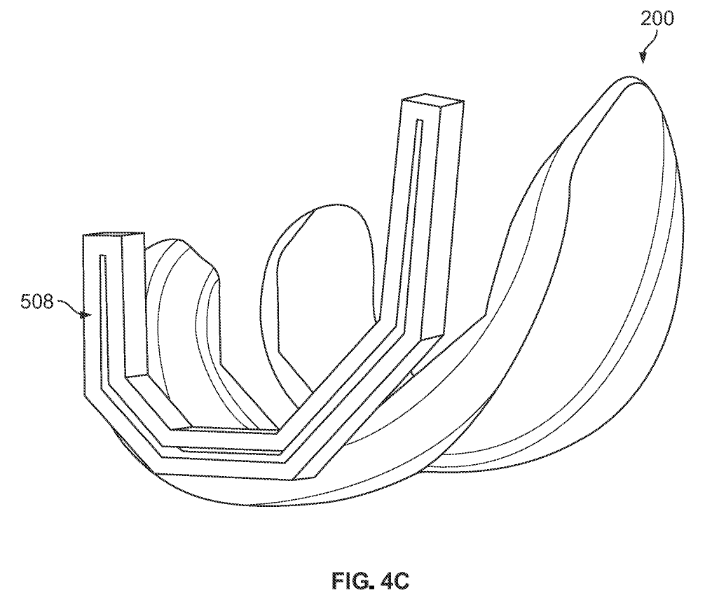

In another embodiment, a method for preparing a bone for receiving an implant using system 100 includes the additional use of an extra-medullary (EM) attachment 500, as shown in FIGS. 4A-4B. EM attachment 500 includes an outer frame configured to receive shell 200, as described further below. EM attachment 500, as shown in FIG. 4A, receives a shell 200 that includes a plurality of slots through base portion 210, while EM attachment 500, as shown in FIG. 4B, receives a shell 200 that includes a plurality of slots through base portion 210 and a plurality of slots through posterior legs 220, 222. EM attachment 500 is fixed to a proximal portion of the femur by a plurality of screws 502 threaded through rings 504 of the EM attachment 500 into the femur, e.g., a proximal region of the femur. In one example, EM attachment 500 includes four set screws 502 designed to anchor the EM attachment 500 to the femur in the anterior, posterior, medial and lateral direction, respectively. EM attachment 500 may be oriented similar to that of linear actuators in a hexapod system, such that the set screws 502 allow for adjustments in anterior/posterior, medial/lateral, and internal/external degrees of freedom. Further, adjustable struts 506 of EM attachment 500 allow for proximal/distal, varus/valgus, and flexion/extension degrees of freedom, e.g., by allowing for the adjustment of a length of one or more of the struts 506. Opposite ends of each strut 506 may include a ball that are respectively received within a socket of ring 504 and a socket of shell 200, such that struts 506 connect EM attachment 500 to shell 200. In other embodiments, each strut 506 may instead be connected to ring 504 and shell 200 by respective swivel joints. Once the outer frame of the EM attachment 500 and shell 200 are in a desired position, shell 200 is pinned into place, struts 506 are removed, and a medial cutting block 508 is snapped to the shell 200 or slid into a slot on the shell 200, as shown in FIG. 4C, in preparation for making resections through the slots of the block 508 and into the distal femur 10. Block 508 may be connected to shell 200 with the use of screws or bolts received through openings in both block 508 and shell 200. In other embodiments, different types of snap-fit features may be used to secure block 508 to shell 200, such as lock and tab features comprising a flexible beam of a cantilever with a hook on one end that deflects and snaps into a recess of the other component, torsional features comprising a twisting action to lock components and optionally utilizing a spring-loaded lever, or annular hoop features comprising a circumferential ridge on one component that expands to snap into a mating groove on the other component.

It should be appreciated that any of the devices, systems, kits and methods disclosed herein may be designed, formed and/or used in conjunction with robotic technology. For example, any of the components described herein may be used with robotic surgical systems for performing a surgical procedure for preparing a bone to receive an implant, and specifically for preparing a distal femur to receive a femoral implant during a revision TKA procedure. The components may be manipulated with a robotic system or a robotic arm to rotate or position the component, and to secure the components to bone or another component during a procedure. Further, any or all of the steps described in the methods for preparing a distal femur to receive a femoral implant, including resecting the femur, may be performed using a robotic system. For example, orientation of shell 200 may be recorded and programed into a robotic cutting device for making resections of the distal femur 10 through any of slots 240, 250.

Although the disclosure herein has been described with reference to particular embodiments, it is to be understood that these embodiments are merely illustrative of the principles and applications of the present disclosure. It is therefore to be understood that numerous modifications may be made to the illustrative embodiments and that other arrangements may be devised without departing from the spirit and scope of the present disclosure as defined by the appended claims.

1. A cutting guide for resecting a bone to receive an implant, the cutting guide comprising:
a body configured to be positioned adjacent to the bone;
a slot extending through the body and configured to receive a cutting edge, the cutting edge being configured to resect the bone; and
an opening extending though the body, the opening being configured to receive a rod,
wherein when a rod is disposed in an intramedullary canal of the bone and the opening of the body is passed over the rod, the body is adjustable such that a plane through the slot changes relative to the bone as the body is adjusted,
wherein the plane through the slot is transverse to a central axis extending through a length of the bone,
wherein the cutting guide is configured such that when the body is received on the rod anchored within the bone, the plane represents a path of resection through the bone, and adjustment of the body alters the path of resection through the bone.

2. The cutting guide of claim 1, wherein the body includes a bone facing surface and an articulation surface opposite the bone facing surface.

3. The cutting guide of claim 1, wherein the bone is a femur and the body is configured to be positioned on the femur such that the cutting edge cuts in an anterior-posterior direction.

4. The cutting guide of claim 1, further comprising a hole extending through the body, the hole being configured to receive a pin anchorable to the bone.

5. The cutting guide of claim 4, wherein the hole is configured to be positioned adjacent a proximal end of the body and remote from the opening.

6. The cutting guide of claim 1, further comprising a reference slot extending through the body and configured to receive the cutting edge for resecting the bone, wherein the reference slot is configured to be positioned adjacent an anatomical point on the bone such that the anatomical point is within a reference plane parallel to a plane through the slot.

7. The cutting guide of claim 6, wherein the bone is a femur and the anatomical point is a medial epicondyle of the femur.

8. The cutting guide of claim 1, wherein the body comprises a flexible material, the body configured to flex as the body is adjusted relative to the bone.

9. A system comprising:
the cutting guide of claim 1; and
the rod configured to flex sufficiently to alter an alignment of the rod when disposed in an intramedullary canal of a long bone.

10. A cutting guide for resecting a bone to receive an implant, the cutting guide comprising:
a body configured to be positioned adjacent to the bone;
a slot extending through the body and configured to receive a cutting edge, the cutting edge being configured to resect the bone; and
an opening extending though the body, the opening being configured to receive a rod,
wherein when the rod is disposed in an intramedullary canal of the bone and the opening of the body is passed over the rod, a portion of the body is rotatable in a sagittal plane and about a central longitudinal axis extending through a length of the bone such that a cutting plane through the slot changes relative to the bone as the body is rotated.

11. The cutting guide of claim 9, wherein the body comprises a flexible material, the body configured to flex as the body is rotated.

12. A method for preparing a bone for receiving an implant, the method comprising: reaming a hole through the bone;
anchoring a rod within the hole;
positioning an opening of a cutting guide over the rod and onto an end portion of the bone such that cutting guide is in a first orientation relative to the bone; and
moving the cutting guide and a portion of the rod positioned through the opening relative to the bone into a second orientation relative to the bone,
wherein in the first orientation a slot of the cutting guide is coincident with a first plane, and in the second orientation the slot of the cutting guide is coincident with a second plane, each of the first plane and the second plane being defined relative to the bone, and
wherein the first plane defines a first cutting path through the bone and the second plane defines a second cutting path through the bone, the first plane and second plane being at different slopes relative to the bone.

13. The method of claim 12, wherein the bone is a femur and the hole through the bone is an intramedullary canal.

14. The method of claim 12, further comprising, after the moving step, comparing the first cutting path through the bone for the cutting guide in the first orientation and the second cutting path through the bone in the second orientation and determining a desired cutting path through the bone.

15. The method of claim 14, wherein an articular surface of the cutting guide includes a reference slot, a third plane passing through the reference slot being parallel to the first and second planes passing through the slot in the first and second orientations, respectively.

16. The method of claim 12, further comprising, after the moving step, anchoring the cutting guide to the bone with a pin extending through a pin hole of the cutting guide.

17. The method of claim 12, further comprising, after the moving step, cutting a portion of the bone through the slot of the cutting guide when the cutting guide is in the first or second orientation.

18. The method of claim 12, wherein the bone is a first bone, and the method further comprises, after the moving step, assessing a flexion space between the first bone and a second bone in the first orientation and in the second orientation, wherein the first bone is a femur and the second bone is a tibia.

19. The method of claim 18, wherein assessing the flexion space includes determining a cutting guide orientation having a minimal flexion space between the femur and the tibia.

20. The method of claim 12, wherein moving the cutting guide further comprises internally and/or externally rotating the cutting guide about a longitudinal axis of the rod.
